(19) United States
(12) Patent Application Publication
(10) Pub. No.: US 2026/0048018 A1
(43) Pub. Date: Feb. 19, 2026

(54) PROCESS FOR THE MANUFACTURE OF A SOLID PHARMACEUTICAL ADMINISTRATION FORM

(71) Applicant: Merck Patent GmbH, Darmstadt (DE)

(72) Inventors: Malte Bogdahn, Babenhausen (DE); Nadine Gottschalk, Darmstadt (DE); Simon Geissler, Neumuenster (DE); Julian Quodbach, Duesseldorf (DE)

(21) Appl. No.: 19/101,659

(22) PCT Filed: Aug. 14, 2023
(86) PCT No.: PCT/EP2023/072357

§ 371 Date: Feb. 6, 2025

(30) Foreign Application Priority Data
Aug. 18, 2022 (EP) 22190883.3

(51) Int. Cl. A61K 9/20; A61J 3/06; A61K 31/496; B33Y 10/00; B33Y 80/00

(52) U.S. Cl. CPC A61K 9/2095; A61J 3/06; A61K 9/2027; A61K 31/496; B33Y 10/00; B33Y 80/00

(57) ABSTRACT

The present invention relates to a process for the preparation of a solid pharmaceutical administration form comprising an amorphous solid dispersion using a 3D printing process. The process is a printing process that allows the production of a solid pharmaceutical solid administration form comprising an amorphous solid dispersion in an easy and flexible manner and the possibility to achieve fast disintegrating dosage forms with high drug loads.

This application is a National Stage Entry under 35 U.S.C. § 371 of International Application No. PCT/EP2023/072357, filed Aug. 14, 2023, which claims the benefit of priority of European Application. No. 22190883.3, filed Aug. 18, 2022, the contents of each of which are incorporated herein by reference in their entireties.

The present invention relates to a process for the preparation of a solid pharmaceutical administration form using a powder-based 3D printing process. The process allows the production of solid pharmaceutical administration forms, wherein the active ingredient is present as an amorphous solid dispersion in a polymeric matrix, in an easy and flexible manner and in conformity with the high-quality standards required for the production of pharmaceuticals.

Many of the recently developed active pharmaceutical ingredients (APIs) suffer from poor aqueous solubility, which leads to incomplete dissolution throughout the gastro-intestinal (GI) tract, resulting in low and variable bioavailability. A significant number of new drug candidates fail during development due to poor bioavailability, with numbers increasing. Therefore, advancement in innovative approaches to overcome this important formulation challenge is critically needed, making the development of new drug delivery systems highly desirable.

In this amorphous form, compounds exhibit higher dissolution rates when compared to their crystalline state, especially when the solubility is limited by high lattice energy. This in turn increases oral bioavailability, as shown by Mellaerts et al. (Eur J Pharm Biopharm 69: 223-230, 2008). 3D printing techniques offer new possibilities in terms of pharmaceutical development. In contrast to standard manufacturing techniques such as tabletting, they enable the production of personalized dosage forms and facilitate clinical trials through easy dose adjustment.

The term 3D printing refers to a process wherein a 3D object is created in a layer-by-layer fashion. However, manufacturing of solid pharmaceutical dosage forms, wherein the active ingredient is present in amorphous form, using the known 3D printing processes is difficult to achieve and may be accompanied with further problems as discussed hereinafter.

Fused Deposition Modeling (FDM) or Fused filament fabrication (FFF) is a process that uses a continuous filament of a thermoplastic material for 3D printing. For manufacturing of pharmaceutical dosage forms API containing filaments are produced by Hot Melt Extrusion (HME), which are then fed into a heated printer nozzle and the softened material is deposited layer-by-layer to create the pharmaceutical dosage form. FDM printed dosage forms consist of solidified dense melt, which dissolves mainly by erosion. Although porous systems are possible, the degree of porousness is limited due to the poor resolution of the printers. Further, recrystallization of active ingredient may occur during the heating at the printing step. High drug loads present a challenge in FDM, as they can impair the mechanical properties of the filaments, which negatively impacts their printability. Further, the second heating step, which is required to soften the filament for printing, is able to trigger degradation of the API.

Direct powder extrusion (DPE) is a 3D printing process, wherein a powder blend is melted and printed in one step into tablets and which avoids double heating and the process of filament production. However, DPE is prone to de-mixing of powder components in the powder blend during printing, thus leading to an impaired content uniformity of the resulting administration forms. Further, as in the case of FDM, the printed administration forms also suffer from poor disintegration due to its dense structure. In addition, manufacturing of an amorphous solid can be more difficult as transition times in the hot end (the heating area above the nozzle in the print head) are short and installed screws may not provide sufficient mechanical energy to amorphize the API. Extrusion processes are very sensitive to variations of mass flow, thus, altering the printing speed in DPE may lead to considerable changes of the properties of the melt or the solidified product.

Another 3D printing technique is selective laser sintering (SLS) where the 3D printed object is manufactured by creating a powder layer and fusion of the powder particles present in the powder layer by a laser. This technique can be used to produce porous dosage forms. The laser has the possibility to amorphize the API in situ, but full amorphization of the API is difficult. The production of larger scales of tablets is limited by the technical conditions of laser printers. Furthermore, due to the very focused alignment of the laser, SLS can lead at certain points of the printed material to very high temperatures, which can have a detrimental effect on the stability of the API and/or other printing material.

Powder binder jetting or drop-on-powder printer is an easy scalable 3D printing technique, which uses a liquid to fuse powder particles. It is used for the production of high dose and fast disintegrating dosage forms. It is currently used for well soluble APIs, where the API is embedded in the powder bed. Manufacturing of amorphous solid dispersion for poorly soluble APIs can be achieved, when the API is incorporated in the ink through fast solvent evaporation similar to spray-drying. This approach can result in amorphous samples, but the drug loads are low. The formulation development of high dose and poorly soluble APIs into fast disintegrating dosage forms represents a challenge.

As set forth above the existing 3D printing processes that are usable for the preparation of solid pharmaceutical administration forms exhibit several disadvantages which hinder their broad applicability. Therefore, there is a strong demand for a 3D printing process that allows the production of solid dosage forms, which is not afflicted with such disadvantages. Such 3D printing process shall lead to solid dosage forms, wherein the active ingredient is present in an amorphous form, shall allow the manufacture of such dosage forms also with active ingredients having a low solubility in biorelevant media and shall also allow the manufacture of such dosage forms having a high content of active ingredient even if the active ingredient exhibits a low solubility in biorelevant media. The present invention provides a process that meets such requirements.

The process of the present invention is a process for the manufacture of a solid pharmaceutical administration form comprising an active ingredient comprising the steps
(a) preparing a powder comprising particles of an amorphous solid dispersion of an active ingredient in a polymeric matrix;
(b) spreading the powder prepared by step (a) across the manufacturing area;
(c) jet printing a medium onto the powder whereby such medium is suitable to provide binding of the powder;
(d) spreading a layer of powder prepared by step (a) onto the surface of powder obtained after performing step (c) and subsequently performing step (c);
(e) repeating step (d) as often as needed to build up the solid pharmaceutical administration form;
(f) separating the solid pharmaceutical administration form from the powder bed.

The process can be run on a 3D printer composed of a pair of horizontal X-Y axes that are suspended over a vertical piston, providing control over three directions of motion and that is equipped with a jet head as known from ink jet printing technology. A suitable jet head may work for example according to the Continuous Inkjet principle (fluid is pressurized and expelled in a continuous stream of droplets), or the drop-on-demand principle (fluid is expelled from the jet nozzle one drop at a time).

For manufacture of solid pharmaceutical dosage form powder is spread onto a mounting plate to create a powder bed, the medium is precisely distributed over predefined areas of the powder bed through by either moving a jet head over the powder bed or moving the powder bed under a fixed jet head. After lowering the mounting plate by a fixed distance, a layer of powder is spread, and the process is repeated. Instead of lowering the mounting plate the spreading means can be raised by a fixed distance.

The process described above, wherein a medium suitable to provide binding of the powder is jet-printed to the powder bed, is known as drop-on-powder (DoP) technique. It belongs to the powder-based 3D printing techniques and can be described as an in-situ wet granulation, where small ink or binder droplets are jetted on thin powder layers resulting in fusion of powder particles. The iterative process of powder spreading and ink application proceeds until the 3D objects is printed.

The term “solid pharmaceutical administration form”, as used herein, means any pharmaceutical formulation that is solid and provides a dosage unit of an active pharmaceutical ingredient that can be administered to a patient by any way of application such as oral, rectal, vaginal, implantation. The solid pharmaceutical administration form can have any shape adapted to the application requirements, e.g. round, oval, rod like, torpedo shaped etc. Examples of solid pharmaceutical administration forms are tablets, pills, caplets, suppositories, implants. Preferably, the solid pharmaceutical administration form is a tablet.

The term “active ingredient”, as used herein, means any ingredient that provides a pharmacological or biological effect when applied to a biological system. The active ingredient may be a pharmaceutical drug, biological matter of viral or living origin. Examples of an active ingredient that may be used in the process of the present inventions are hydrocortisone, prednisone, budesonide, methotrexate, mesalazine, sulfasalazine, amphotericin B, fenofibrate, carbamazepine, ibuprofen, glibenclamide, dipyridamole, itraconazole, celecoxib, haloperidol, indomethacin, posaconazole, ketoconazole.

The term “solid dispersion”, as used herein, refers to a drug substance, which is dispersed or distributed in a dispersion medium. In the present invention the dispersion medium is a polymer, which forms a polymeric matrix. Based upon the possible combinations of the drug substance and polymer physical states, the drug substance can be either crystalline or amorphous and the polymeric matrix can also be crystalline and amorphous, resulting in four possible combinations: crystalline drug substance-crystalline polymer (solid suspension); amorphous drug substance-amorphous polymer; crystalline drug-amorphous polymer; and amorphous drug-crystalline polymer.

The term “amorphous solid dispersion” (ASD), as used herein, refers to a dispersion wherein at least the active ingredient is present in substantially amorphous form. Preferably both, the active ingredient and the polymer are present in substantially amorphous form. With respect to the active ingredient the term “substantially amorphous form” means that at least 80 percent by weight, typically at least 85 percent by weight, preferably at least 90 percent by weight, more preferably at least 95 percent by weight, still more preferably at least 96 percent by weight, still more preferably at least 97 percent, more preferably at least 98 percent by weight, more preferably at least 99 percent by weight, more preferably at least 99.9 percent by weight, more preferably all active ingredient, is present in amorphous form.

The term “amorphous”, as used herein, relates to the non-crystalline form of a solid. Amorphous solids generally possess crystal-like short-range molecular arrangements, i.e. no long-range order of molecular packing found in crystalline solids. The solid form of a solid in the solid dispersion may be determined by polarized light microscopy, X-ray powder diffraction, differential scanning calorimetry or other techniques known to those of skill in the art. The amorphous form of the active ingredient in the solid dispersion can usually be identified by a distinctive broad X-Ray powder diffraction pattern, whereas crystalline solids lead to specific isolated peaks. The amorphous form can exist in two states: one rubbery state and one glass state, where one state converts to the other one at the glass transition temperature (Tg).

The term “polymeric matrix”, as used herein, describes a three-dimensional solid that is formed by one or more than one polymer. In the powder used in present invention the polymeric matrix is used to embed the active ingredient. Further compounds such as, for example, one or more further active ingredients or other excipients, can be incorporated, such as dissolved or dispersed, in such polymeric matrix.

Preparing of the powder in step (a) comprises the preparation of an amorphous solid dispersion of an active ingredient in a polymeric matrix, and, if needed, reduction of its size to a particle size that is usable in the process, which can be performed by using appropriate processes known in the art such as, for example, milling. If the powder comprises further material the amorphous solid dispersion particles are mixed with such material to create the powder used in the further steps. If no further material is needed the solid dispersion particles represent the powder prepared in step (a), which are used in the further steps.

Powders suitable for performing the further steps of the process usually have a d50 particle diameter from about 1 μm to about 200 μm, preferably from about 10 μm to about 100 μm, more preferably from about 30 μm to about 70 μm. d50 values referred to herein relate to the particle diameter in micrometres that splits the distribution with half above and half below this diameter. The d50 is the median for a volume distribution and is often also designated Dv50 (or Dv0.5). The d50 values referred to herein are those that can be measured by laser diffraction using a Malvern Mastersizer 2000.

The term “spreading” as used herein means a process where a planar layer of powder is applied to a planar ground. Spreading of powder can be achieved by using means that are suitable to create a planar layer of powder. Examples of such means are a doctor blade or a roller that can be moved in parallel to planar ground such as a mounting area or an existing powder layer to distribute the powder from a reservoir across the planar ground. By the use of a roller a certain level of compaction can be obtained, which may be advantageous for the manufacture of the solid pharmaceutical dosage form.

As used herein, “a” or “an” shall mean one or more. As used herein when used in conjunction with the word “comprising,” the words “a” or “an” mean one or more than one. As used herein “another” means at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−1-3% of the recited value) that one of ordinary skill in the art would consider equivalent to the recited value (e.g., having the same function or result). In some instances, the term “about” may include numerical values that are rounded to the nearest significant figure.

As used herein, “jet printing” refers to a process where a medium is distributed to the powder bed by ejecting droplets of medium at high speed towards and onto the powder bed. Ejection of droplets can be performed with utmost precision to predefined target place. By managing size and/or amount of droplets and specific target place the exact placement on and penetration depth in a substrate can be precisely controlled. Jet printing is well-known from inkjet printing technology but in contrast to this technology the medium that is printed in the process of the present invention is not an ink for printing of images but a medium, that contains materials that are usable for printing of solid pharmaceutical administration forms.

The amorphous solid dispersion of an active ingredient in a polymeric matrix, which is present as particles in the powder prepared according to step (a) of the process, can be prepared using any method known in the art that is feasible for the preparation of an amorphous solid dispersion of an active ingredient in a polymeric matrix. According to suitable embodiments the amorphous solid dispersion is prepared using hot melt extrusion, co-precipitation or spray drying. Thus, the invention is also directed to a process for the manufacture of a solid pharmaceutical administration form, wherein the amorphous solid dispersion of an active ingredient in a polymeric matrix, which is present as particles in the powder prepared by step (a) is prepared using hot melt extrusion, co-precipitation or spray drying. Preparation of the powder by hot melt extrusion is especially preferred.

The term “hot melt extrusion”, as used herein, refers to a process whereby two or more components are mixed using high shear mixing at a controlled temperature. When used for the preparation of the powder in accordance to step (a) of the process, hot melt extrusion comprises mixing together an active ingredient and at least one polymer until a soft mass is produced. The mixing of the active ingredient and a polymer can happen before, during or after the formation of the soft mass. For example, the required ingredients to produce the soft mass may be mixed initially and then extruded or may be simultaneously mixed and melt extruded. Finally, the hot melt is homogenized so as to disperse or embed the active ingredient into the polymer.

The process of hot melt extrusion may be carried out by using conventional extruders that are known in the art. Suitable extruders include, but are not limited to, single-screw extruders, intermeshing screw extruders or else multi-screw extruders, preferably twin-screw extruders, which can be co-rotating or counter-rotating and, optionally, be equipped with kneading mixing and/or conveying elements. The working temperature for preparing hot melt extrusion typically depends on the API and polymer properties as well as extruder type and screw configuration. The extrudate obtained by the hot melt extrusion may be further processed, for example by milling, to provide a powder having a size and shape usable in the process.

The term “co-precipitation”, as used herein, refers to a process whereby two or more solid components are dissolved in a common solvent and precipitated by rapid mixing with a common anti-solvent. The anti-solvent is miscible with the common solvent. Rapid co-precipitation of an active ingredient with a polymer may yield a suspension of amorphous particles that may be further washed and dried to a powder.

The term “spray drying” as used herein refers, in principle, to a solvent extraction process. The constituents of the product to be obtained are dissolved/dispersed in a liquid and then fed, for example by using a peristaltic pump, to an atomiser of a spray-dryer. A suitable atomizer, which can be used for atomization of the liquid, include nozzles or rotary discs. With nozzles, atomization occurs due to the action of the compressed gas or pressurized liquid, while in case of using rotary discs atomization occurs due to the rapid rotation of the disc. In both cases, atomization leads to disruption of the liquid into small droplets into the drying chamber, wherein the solvent is extracted from the aerosol droplets and is discharged out, for example through an exhaust tube to a solvent trap.

The medium used for jet printing is a liquid. The term “liquid”, as used herein, refers to solvents, that are fluid at ambient temperature (about 25° C.). Examples of liquids are water, organic solvents, such as ethanol, or mixtures of both, whereby the organic solvent may be soluble with one another or not. Thus, the invention is also directed to a process for the manufacture of a solid pharmaceutical administration form, wherein the medium used for jet printing in step (c) is a liquid.

The liquid may further contain auxiliaries, which may be dissolved, suspended or emulsified in the liquid. Auxiliaries that may be used are surfactants, e.g. to improve spreading or wetting of particles in the powder bed. Further examples of auxiliaries include viscosity modifiers, e.g. glycerol to enable jet printing by preventing excessive wetting of the nozzle plate, or by controlling the flow of the liquid through the channels and nozzles of the jet head; agents to control the hydrophilicity or hydrophobicity of the ink, e.g. co-solvents, such as ethanol, butanol, diethylene glycol, polyethylene glycol, dimethyl sulfoxide, hexane, to improve spreading or wetting of particles in the powder bed; humectants, e.g. glycerol or propylene glycol to prevent nozzle clogging by ink evaporation; film formers, sometimes called binders or resins, to control the spreading of the ink on the substrate, and to prevent bleeding or smearing of the ink on the substrate; dyes or pigments; and defoamers.

The liquid that is jet printed to the powder in step (c) itself provides binding of the powder so that the presence of a binder as it is known by process of binder jetting is not necessary. However, in some instances it may be advantageous that the powder that is spread across the manufacturing area comprises a binding material, that upon activation by the medium jet printed to the powder provides additional binding. Thus, the invention is also directed to a process, wherein the powder comprises a binding material. If the powder comprises a binding material, it is present in the powder in physical admixture with the other powder particles.

In some instances, if the binding material is a polymer, the binding material present in the powder may be the same material as this used as matrix material for the amorphous solid dispersion.

When the medium is jet printed to the powder binding of the powder is provided by partial dissolving and fusing of the polymeric matrix and activating of the binding material, if present in the powder. Therefore, the invention is also directed to a process for the manufacture of a solid pharmaceutical administration form, wherein the medium is a fluid liquid which partly dissolves the polymeric matrix and/or the binding material present therein.

Partly dissolving of the polymeric matrix means that part of the polymeric matrix of particles is dissolved to some extent and softened, thus inducing sticking and/or partially fusing of particles that are in close contact to each other and building up a porous structure of adhered and/or fused particles.

Advantageously, the medium to be jet printed onto the powder that is used to cause sticking and adhering of the particles comprises at least one volatile solvent. A volatile solvent is a liquid that easily vaporizes into a gas at room temperature (about 25 degrees Celsius) and at atmospheric pressure (about 760 mmHg), such as, for example, an organic solvent like methanol and ethanol. Upon contact with the powder comprising the polymeric matrix and causing partly dissolving and/or fusing of the polymeric matrix of the powder particles adjacent to each other the volatile solvent disappears by evaporation thereby causing re-solidification of the polymer matrix present in the powder and sticking of the powder.

The physicochemical properties of the medium needed to cause sticking and/or partly fusing of the matrix polymer present in the powder, e.g. its ability to dissolve the polymer of the polymeric matrix and/or its volatility can be easily adapted to the specific demands of a specific polymer as well as to the requirements for the execution of the printing process by using different volatile solvents and/or admixing of volatile solvents alone or together with non-volatile solvents. Thus, the invention is further directed to a process wherein the medium comprises or consists of one or more volatile solvent(s) alone or in admixture with one or more non-volatile solvent(s). A non-volatile solvent is a liquid that does not easily vaporize into a gas at room temperature (about 25 degrees Celsius) and at atmospheric pressure (about 760 mmHg) and that has a vapor pressure equal or less to that of water at such conditions.

Suitable volatile solvents that are usable as medium in the process of the present invention are methanol, ethanol, propanol, 2-propanol and acetone; suitable non-volatile solvents are water, N-methyl-2-pyrrolidone, N-ethyl-2-pyrrolidone, dimethylformamide, dimethylacetamide and dimethylsulfoxide. Thus, the present invention is also directed to process for the manufacture of a solid pharmaceutical administration, wherein the volatile solvent is selected from the group consisting of methanol, ethanol, propanol, 2-propanol and acetone and the non-volatile solvent is selected from the group consisting of water, N-methyl-2-pyrrolidone, N-ethyl-2-pyrrolidone, dimethylformamide, dimethylacetamide and dimethylsulfoxide. Methanol and ethanol are especially preferred.

In some instances, the medium jet printed to the powder may further comprise a binding material. Thus, the invention is further directed to a process for the manufacture of a solid pharmaceutical administration form, wherein the medium is a liquid and comprises a binding material. If the medium comprises a binding material such binding material is present in dissolved or dispersed form. Preferably it is present in dissolved form.

The term “binding material”, as used herein, refers to a material that provides binding or sticking of particles. When applied onto the powder bed, the particles, that come into contact with the binding material, adhere to each other thereby generating a solid composed of particles, which are attached to each other. In the present invention the binding material provides cohesion and strength to the solid preparation.

Binding materials which can be employed in the present invention are, for example, lactose, sorbitol, mannitol, xylitol, maltitol, glucose, fructose, sucrose, sucrose fatty acid esters (e.g. sucrose stearate, sucrose palmitate), sorbitan esters (e.g. Span®), glycerol fatty acid esters (e.g. glycerol monostearate), fatty acids, fatty alcohols (solid at room temperature), esters of fatty acids with fatty alcohols as well as polymers such as polyvinylpyrrolidone, polyvinyl alcohol, polyvinyl acetate, a vinylpyrrolidone-vinyl acetate copolymer, polyethylene glycol, a starch, such as maize starch or pre-gelatinized starch, a cellulose derivative, such as hydroxypropyl methylcellulose, hydroxypropyl cellulose, ethyl cellulose, hydroxypropyl methylcellulose acetate succinate or microcrystalline cellulose, a copolymer of acrylic or methacrylic acid and an acrylic or methacrylic ester, preferably a copolymer of methacrylic acid and a methacrylate or a acrylate, such as, for example Poly(methacrylic acid-co-methyl methacrylate) (1:1) (e.g. Eudragit® L 100), Poly(methacrylic acid-co-methyl methacrylate) (1:2) (e.g. Eudragit® S 100) or Poly(methacrylic acid-co-ethyl acrylate) (1:1) (e.g. Eudragit® L 100-55), a copolymer of ethyl acrylate, methyl methacrylate and a low content of methacrylic acid ester with quaternary ammonium groups, such as, for example, Poly(ethyl acrylate-co-methyl methacrylate-co-trimethylammonioethyl methacrylate chloride) 1:2:0.2 (e.g. Eudragit® RL) or Poly(ethyl acrylate-co-methyl methacrylate-co-trimethylammonioethyl methacrylate chloride) 1:2:0.1 (e.g. Eudragit® RS), a copolymer of dimethylaminoethyl methacrylate, butyl methacrylate, and methyl methacrylate, such as, for example, Poly(butyl methacrylate-co-(2-dimethylaminoethyl) methacrylate-co-methyl methacrylate) (2:1:1) (e.g. Eudragit® E PO), preferably hydroxypropyl methylcellulose acetate succinate or polyvinyl alcohol, more preferably hydroxypropyl methylcellulose acetate succinate. Therefore, the present invention is as well directed to a process for the manufacture of a solid pharmaceutical administration form, wherein the binding material comprises or consists of lactose, sorbitol, mannitol, xylitol, maltitol, glucose, fructose, sucrose, sucrose fatty acid esters (e.g. sucrose stearate, sucrose palmitate), sorbitan esters (e.g. Span®), glycerol fatty acid esters (e.g. glycerol monostearate), fatty acids, fatty alcohols (solid at room temperature), esters of fatty acids with fatty alcohols and polymers such as polyvinylpyrrolidone, polyvinyl alcohol, polyvinyl acetate, a vinylpyrrolidone-vinyl acetate copolymer, polyethylene glycol, a starch, such as maize starch or pre-gelatinized starch, a cellulose derivative, such as hydroxypropyl methylcellulose, hydroxypropyl cellulose, ethyl cellulose, hydroxypropyl methylcellulose acetate succinate or microcrystalline cellulose, a copolymer of acrylic or methacrylic acid and an acrylic or methacrylic ester, preferably a copolymer of methacrylic acid and a methacrylate or a acrylate, such as, for example Poly(methacrylic acid-co-methyl methacrylate) (1:1) (e.g. Eudragit® L 100), Poly(methacrylic acid-co-methyl methacrylate) (1:2) (e.g. Eudragit® S 100) or Poly(methacrylic acid-co-ethyl acrylate) (1:1) (e.g. Eudragit® L 100-55), a copolymer of ethyl acrylate, methyl methacrylate and a low content of methacrylic acid ester with quaternary ammonium groups, such as, for example, Poly(ethyl acrylate-co-methyl methacrylate-co-trimethylammonioethyl methacrylate chloride) 1:2:0.2 (e.g. Eudragit® RL) or Poly(ethyl acrylate-co-methyl methacrylate-co-trimethylammonioethyl methacrylate chloride) 1:2:0.1 (e.g. Eudragit® RS), a copolymer of dimethylaminoethyl methacrylate, butyl methacrylate, and methyl methacrylate, such as, for example, Poly(butyl methacrylate-co-(2-dimethylaminoethyl) methacrylate-co-methyl methacrylate) (2:1:1) (e.g. Eudragit® E PO), preferably hydroxypropyl methylcellulose acetate succinate or polyvinyl alcohol, more preferably hydroxypropyl methylcellulose acetate succinate.

Polymers usable as matrix polymers for the preparation of the powder comprising an amorphous solid dispersion of an active ingredient in a polymeric matrix are vinylpyrrolidone-vinyl acetate copolymer (PVP-VA), polyvinylpyrrolidone (PVP), polyvinyl alcohol (PVA), hydroxypropyl methylcellulose acetate succinate (HPMCAS), (Eudragit® L100-55) and poly(methacrylic acid-co-methyl methacrylate) (Eudragit® L and Eudragit® S), polyvinyl caprolactam-polyvinyl acetate-polyethylene glycol graft copolymer (PVCap-PVAc-PEG), (Soluplus®), hydroxypropyl methylcellulose phthalate (HPMCP), cellulose acetate phthalate (CAP), polyvinyl acetate phthalate (PVAP), cellulose acetate trimellitate (CAT) or hydroxypropyl methylcellulose acetate trimellitate (HPMCAT). Hence the present invention is also directed to a process for the manufacture of a solid pharmaceutical administration form, wherein the polymeric matrix comprises or consists of a vinylpyrrolidone-vinyl acetate copolymer (PVP-VA), polyvinylpyrrolidone (PVP), polyvinyl alcohol (PVA), hydroxypropyl methylcellulose acetate succinate (HPMCAS), (Eudragit® L100-55) and poly(methacrylic acid-co-methyl methacrylate) (Eudragit® L and Eudragit® S), polyvinyl caprolactam-polyvinyl acetate-polyethylene glycol graft copolymer (PVCap-PVAc-PEG), (Soluplus®), hydroxypropyl methylcellulose phthalate (HPMCP), cellulose acetate phthalate (CAP), polyvinyl acetate phthalate (PVAP), cellulose acetate trimellitate (CAT) or hydroxypropyl methylcellulose acetate trimellitate (HPMCAT).

In some instances, depending on the type and quantity of the medium jet printed to the powder, especially the volatility of the liquid, as well as from the physicochemical properties of the powder after jet printing of the medium to the powder according to step (c), it may be necessary to wait for some time to allow the liquid to evaporate prior to proceeding the next steps. In such instances it may be desirable to accelerate the evaporation of the liquid, which would allow to run the process faster. In such instances, a drying step may be introduced after performing step (c) and/or step (d). Accordingly, the present invention is further directed to a process for the manufacture of a solid pharmaceutical administration, wherein a drying step is performed after performing step (c) and/or step (d).

According to a suitable embodiment the drying step is performed by using heating, lowering air pressure or convection. As each of such measures facilitates evaporation on its own, each of such measures can be combined with one or more of the other measures to achieve an additive effect and to speed up the drying step. Hence, the invention is as well directed to process for the manufacture of a solid pharmaceutical administration, wherein the drying step comprises heating, low (air) pressure, and/or convection. An appropriate low air pressure is an air pressure below atmospheric pressure, for example an air pressure in the range from 100 to 80000 Pa, preferable in the range from 5000 to 50000 Pa. Convection may be applied, for example, by an air blower. An example of an embodiment of a drying step, wherein heating is combined with convection is blowing heated air to the powder bed by use of an air blower.

Heating can be applied by infrared irradiation, a hot gas flow and/or heated surfaces. Thus, the invention is also directed to a process for the manufacture of a solid pharmaceutical administration form, wherein heating is applied by infrared irradiation, a hot gas flow and/or heated surfaces. The gas of the hot gas flow can be any gas of one element or chemical compound such as, for example, nitrogen or carbon dioxide, or a mixture of elements and chemical compounds gases, such as air. Heated surfaces can be provided, for example, by heating the mounting plate or heating part or all of the encasement of the 3D printer used for running the process.

In principle, a solid pharmaceutical administration form for any way of application such as oral, rectal, vaginal, implantation can be manufactured with the process as described herein. However, the process is especially suitable for the manufacture of a solid pharmaceutical administration form for oral use. Thus, an advantageous embodiment of the invention described herein is directed to a process for manufacture of a solid pharmaceutical administration form, wherein the pharmaceutical administration form is for oral use.

As the active ingredient is present in an amorphous solid state its dissolution in biorelevant media is increased. Thus, the process of the invention is especially suitable for manufacture of immediate release formulations. Accordingly, a preferred embodiment of the invention is directed to the process for manufacture of a solid pharmaceutical administration form, wherein the pharmaceutical administration form provides immediate release of the active ingredient.

The term “immediate release”, as used herein, means that the majority of the active pharmaceutical ingredient is released quickly from the pharmaceutical administration form. Preferably, at least 80 percent of the active is released within 30 minutes from administration, more preferably within 15 minutes. The release of the active ingredient from the pharmaceutical administration form (the dissolution) is measured in pH 6.8 buffer or 0.1 N HCl using conventional dissolution testing in line with standard dissolution tests described in applicable Pharmacopoeias (e.g. Chapter 711 USP).

In some instances, after its separation from the powder bed in accordance to step (f) of the process, the solid pharmaceutical administration form may still contain some residual liquid not evaporated so far that needs to be removed prior to further handling, e.g. to avoid physical damage. In such instances it could be necessary to remove the remaining liquid from the solid pharmaceutical administration form with a subsequent drying step. Thus, the invention is also directed to a process for manufacture of a solid pharmaceutical administration form, wherein a drying step is performed after step (f). Such drying step comprises heating, low (air) pressure, and/or convection.

In the following, the present invention will be described by reference to exemplary embodiments thereof, which shall not be regarded as limiting the invention.

### EXAMPLES

### Printing Apparatus

The following examples were manufactured with an apparatus comprising a powder bed which can be moved in x and y direction of the machine and serves as manufacturing area for the described objects. A print head assembly is located over the powder bed. The assembly comprises one modified HP C6602 ink jet cartridge. The cartridge is connected to an electronic circuit which can activate the nozzles to eject droplets of fluids synchronized to the motions of the powder bed. The cartridge is modified in a way that the ink contained in the stock cartridge can be replaced. Furthermore, a connector is introduced to connect the cartridge to a pressure regulator. A negative pressure of 20 mm H₂O is applied to the ink reservoir of the cartridge. For jet printing a medium comprising a binder the native nozzle pitch of the cartridge was used. At a different position a powder reservoir is mounted over the build plate comprising a powder. The material can be deposited onto the manufacturing area of the powder bed in a controlled manner.

The printing process is controlled via software commands executed in a sequential order. First a thin powder layer is prepared in the powder bed. Afterwards the powder bed is moved under the printing assembly and a liquid material is jet printed onto the surface of the powder bed. After all printing commands and optional dwell times for a specific layer are executed the next layer of particulate material (height: 0.1 mm) is deposited onto the already prepared powder bed surface and liquid material is jet printed onto the new powder layer. The process is repeated until all layers of the objects were printed.

The printing pattern as well as the necessary motions to manufacture a specific object are defined by a software which takes a digital 3-dimensional model and a settings file.

The shape of printed objects can be defined via a digital 3 dimensional model.

Each nozzle of the print head ejected 500 droplets per second maximum.

### Droplet Volume

The Droplet volume of the print heads is measured by printing a defined number of droplets in the cavities of an acrylic 96-well plate. The deposited material is diluted, and concentration of the incorporated dye is determined via UV/VIS spectroscopy. The droplet volume is calculated with the following formula:

$${V}_{d r o p l e t} = \frac{{c}_{d y {e}_{s a m p l e} \times {V}_{s a m p l e}}}{{c}_{dye\_ink} \times {n}_{d r o p l e t s}}$$

### Preparation of Powder Bed

The powder used in the printing process was prepared by hot-melt extrusion. Ketoconazole, copovidone and fumed silica were blended in a tumbler mixer in ratios of 20:79:1 and 40:59:1. Ketoconazole was used as poorly soluble model compound. Extrusion was performed on a co-rotating twin-screw extruder equipped with 11 mm screws. Extrusion was performed above the melting temperature of ketoconazole. Powder blend was gravimetrically fed at 0.2 kg/h. Screw speed was set to 300 rpm. Extrudate strand was pulled using a conveyor belt. Collected extrudate strands were milled using an ultra-centrifugal mill equipped with a sieve (mesh size 200 μm). Milling was performed at 10.000 rpm.

### Glossary

Copovidone: copolymer of 1-vinyl-2-pyrrolidone and vinyl acetate in a ratio of 6:4 by mass (Ph Eur. 11.0 monograph “Copovidone”)
Fumed silica: Colloidal silicon dioxide according to Ph. Eur. 11.0 monograph “Silica, collodial anhydrous”)
Methylene blue: IUPAC name 3,7-bis(Dimethylamino)-phenothiazin-5-ium chloride

### Non-Sink Dissolution

1.2 ml of FaSSIF was prepared from FaSSIF powder (Biorelevant.com Ltd, London, United Kingdom) (L. Klumpp, Dissolution behavior of various drugs in different FaSSIF versions, European Journal of Pharmaceutical Sciences, 2020) according to instructions and heated to 37° C. in Eppendorf caps. After introducing the milled and weighed sample into the medium the Eppendorf cap was vortexed. Before a sample was drawn, the medium was centrifuged and 50 μl of the supernatant was sampled. After sampling the solid fraction was re-suspended by vortexing. The sample was diluted with organic solvent to prevent precipitation of ketoconazole and concentration of ketoconazole in the samples was determined via UPLC.

The drug load of the objects was determined by diluting the medium after the dissolution experiment with organic solvent to solve ketocoanzole comprehensively and concentration of ketocoanzole was determined via UPLC. The mass of API in the printed object was calculated from all drawn samples and the end value.

### Sink Dissolution

Formulation prototypes were analyzed in a dissolution apparatus equipped with paddles according to USP apparatus type 2. Dissolution medium (0.1 N hydrochloric acid) was heated up to 37° C. Paddle speed was set to 100 rpm. Samples were drawn at various timepoints, mixed with an equal volume of organic solvent and analyzed in terms of concentration via UPLC.

### Tensile Strength

The resistance to crushing of tablet was determined according to Ph. Eur. 11.0, 2.9.8, “Resistance to crushing of tablets”. The tensile strength was calculated according to the equation for flat-face compact in Pitt, K. G. and M. G. Heasley (2013), “Determination of the tensile strength of elongated tablets.” Powder Technology 238:169-175.

### Storage Conditions

Formulation prototypes were stored at 40° C. in a desiccator for four weeks.

### Differential Scanning Calorimetry (DSC)

Samples were weighted into aluminum pans and sealed hermetically. Prior to analysis pans were pierced. Samples were heated from 0° C.-180° C. (physical mixture) or 0° C.-200° C. (formulation prototypes) and cooled down to 0° C. at a rate of 10 K/min.

Powder X-Ray Diffraction (pXRD)

Powder X-ray diffraction was performed in Bragg-Brentano geometry. X-rays were generated by a copper anode at 30 KV and 10 mA. Reduction of Kβ radiation was done by utilizing nickel foil. Sample preparation was performed on zero-background holders in a range of 6°-35°. Step size was 0.02 mm. Measurement time per step was set to 6 s in case of formulation prototypes and 1 s in case of physical mixtures.

In the following, the present invention will be further described by reference to exemplary embodiments thereof, which shall not be regarded as limiting the invention.

### Example A

The medium is prepared by mixing ethanol and purified water in a volume ratio of 7:3. A powder bed prepared by hot-melt extrusion and milling comprising 20% (w/w) ketoconazole is used. The medium is jet printed onto the surface of the powder bed with 30 droplets per mm in printing direction. A new layer of powder (0.1 mm) is applied onto the surface of the powder bed and the printing process is repeated for 24 layers in total.

The described process results in objects (shape: cylinder with diameter=10 mm and height=2.4 mm) with a mass of 150.6 mg±6.2 mg (mean±sd, n=75) and drug load of 19.85%+0.09% (mean±sd, n=3) and tensile strength of 0.9 MPa±0.3 MPa (mean±sd, n=10).

### Example B

The medium is prepared by mixing ethanol and purified water in a volume ratio of 7:3. A powder bed prepared by hot-melt extrusion and milling comprising 40% (w/w) ketoconazole is used. The medium is jet printed onto the surface of the powder bed with 30 droplets per mm in printing direction. A new layer of powder (0.1 mm) is applied onto the surface of the powder bed and the printing process is repeated for 24 layers in total.

The described process results in objects (shape: cylinder with diameter=10 mm and height=2.4 mm) with a mass of 157.6 mg+69.7 mg (mean±sd, n=24) and tensile strength of 1.1 MPa±0.2 MPa (mean±sd, n=3).

### Example C

The medium is pure methanol. A powder bed prepared by hot-melt extrusion and milling comprising 20% (w/w) ketoconazole is used. The medium is jet printed onto the surface of the powder bed with 30 droplets per mm in printing direction. A new layer of powder (0.1 mm) is applied onto the surface of the powder bed and the printing process is repeated for 24 layers in total.

The described process results in objects (shape: cylinder with diameter=10 mm and height=2.4 mm) with a mass of 148.5 mg+3.7 mg (mean±sd, n=27) and drug load of 16.1%±0.8% (mean±sd, n=3) and tensile strength of 0.6 MPa±0.2 MPa (mean±sd, n=3).

### Example D

The medium is pure methanol. A powder bed prepared by hot-melt extrusion and milling comprising 40% (w/w) ketoconazole is used. The medium is jet printed onto the surface of the powder bed with 30 droplets per mm in printing direction. A new layer of powder (0.1 mm) is applied onto the surface of the powder bed and the printing process is repeated for 24 layers in total.

The described process results in objects (shape: cylinder with diameter=10 mm and height=2.4 mm) with a mass of 151.9 mg±6.9 mg (mean±sd, n=36) and drug load of 35.7%±1.3% (mean±sd, n=3) and tensile strength of 0.5 MPa±0.1 MPa (mean±sd, n=4).

### Example E

The medium is prepared by mixing ethanol and purified water in a volume ratio of 7:3. A powder bed prepared by hot-melt extrusion and milling comprising 40% (w/w) ketoconazole is used. The medium is jet printed onto the surface of the powder bed with 40 droplets per mm in printing direction.

The described process results in single layer objects.

### Example F

The medium is pure methanol containing methylene blue in a concentration of 0.15 mg/mL. A powder bed prepared by hot-melt extrusion and milling comprising 40% (w/w) ketoconazole is used. The medium is jet printed onto the surface of the powder bed with 40 droplets per mm in printing direction.

The described process results in single layer objects.

### Example G

The medium is pure methanol containing methylene blue in a concentration of 0.15 mg/mL. A powder bed prepared by hot-melt extrusion and milling comprising 40% (w/w) ketoconazole is used. The medium is jet printed onto the surface of the powder bed with 50 droplets per mm in printing direction.

The described process results in single layer objects.

### Example H

The medium is prepared by mixing isopropyl alcohol and purified water in a volume ratio of 9:1 and adding methylene blue in a concentration of 0.16 mg/mL. A powder bed prepared by hot-melt extrusion and milling comprising 40% ketoconazole is used. The medium is jet printed onto the surface of the powder bed with 40 droplets per mm in printing direction.

The described process results in single layer objects.

### Example I

The medium is prepared by mixing isopropyl alcohol and purified water in a volume ratio of 9:1 and adding methylene blue in a concentration of 0.16 mg/mL. A powder bed prepared by hot-melt extrusion and milling comprising 40% (w/w) ketoconazole is used. The medium is jet printed onto the surface of the powder bed with 50 droplets per mm in printing direction.

The described process results in single layer objects.

The invention is illustrated in the Figures.

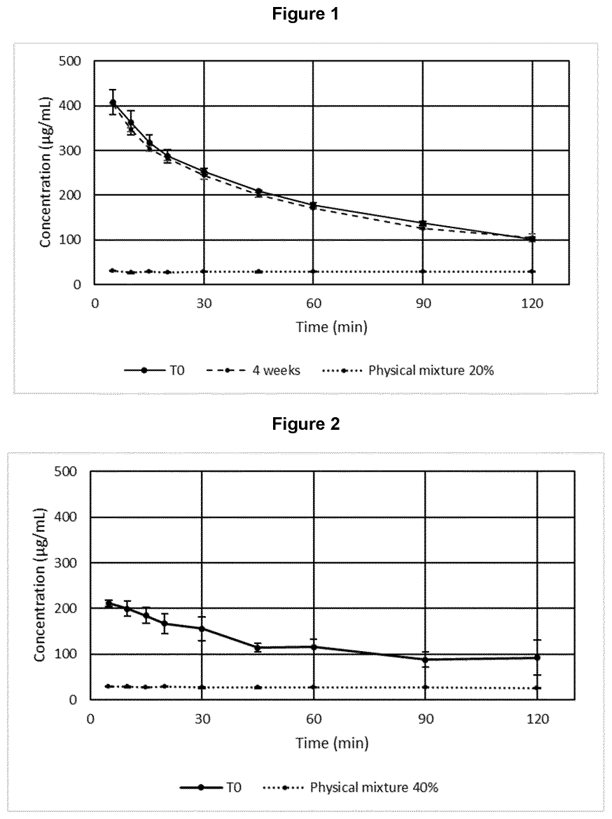

### FIG. 1

FIG. 1 shows the non-sink dissolution curves of formulation prototypes (mean±SD, n=3). Solid line: Example A. Dashed line: Example A after four weeks of storage. Dotted line: crystalline ketoconazole in physical mixture with copovidone.

The examples showed a higher supersaturation of ketoconazole in dissolution medium than a physical mixture of ketoconazole and the polymer. Supersaturation profile remained similar after storage over a period of four weeks at accelerated storage conditions indicating samples were physically stable.

### FIG. 2

FIG. 2 shows the non-sink dissolution curves of formulation prototypes (mean±SD, n=3). Solid line: Example B. Dotted line: crystalline ketoconazole in physical mixture with copovidone.

The examples showed a higher supersaturation of ketoconazole in dissolution medium than a physical mixture of ketoconazole and the polymer.

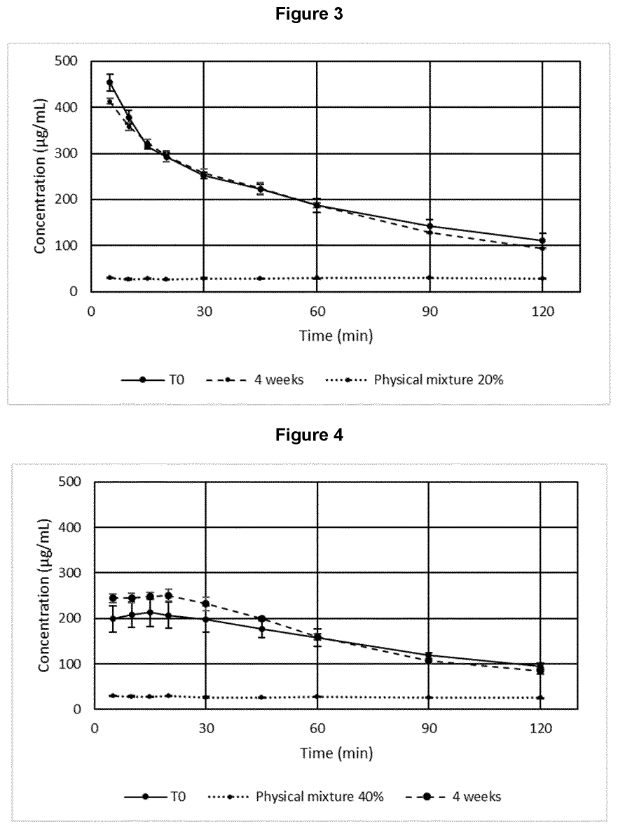

### FIG. 3

FIG. 3 shows the non-sink dissolution curves of formulation prototypes (mean±SD, n=3). Solid line: Example C. Dashed line: Example C after four weeks of storage. Dotted line: crystalline ketoconazole in physical mixture with copovidone.

The examples showed a higher supersaturation of ketoconazole in dissolution medium than a physical mixture of ketoconazole and the polymer. Supersaturation profile remained similar after storage over a period of four weeks at accelerated storage conditions indicating samples were physically stable.

### FIG. 4

FIG. 4 shows the non-sink dissolution curves of formulation prototypes (mean±SD, n=3). Solid line: Example D. Dashed line: Example D after four weeks of storage. Dotted line: crystalline ketoconazole in physical mixture with copovidone.

The examples showed a higher supersaturation of ketoconazole in dissolution medium than a physical mixture of ketoconazole and the polymer. Supersaturation profile remained similar after storage over a period of four weeks at accelerated storage conditions indicating samples were physically stable.

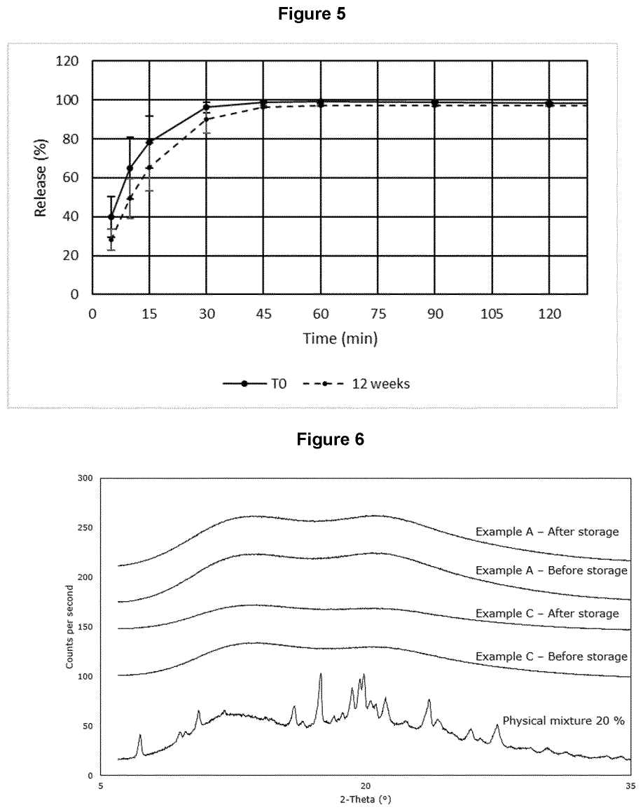

### FIG. 5

FIG. 5 shows the sink-dissolution curves of formulation prototypes from example A (mean±SD, n=3). Solid line: Example A right after preparation. Dashed lines: Example A after twelve weeks of storage.

Example A: This formulation released 80% of ketoconazole in less than 30 min meeting the criteria for immediate release solid oral dosage forms.

### FIG. 6

FIG. 6 shows pXRD measurements of Examples A and C before and after storage compared to a physical mixture containing the polymer and 20% of ketoconazole.

Graphs show that formulation prototypes were amorphous after preparation and after storage.

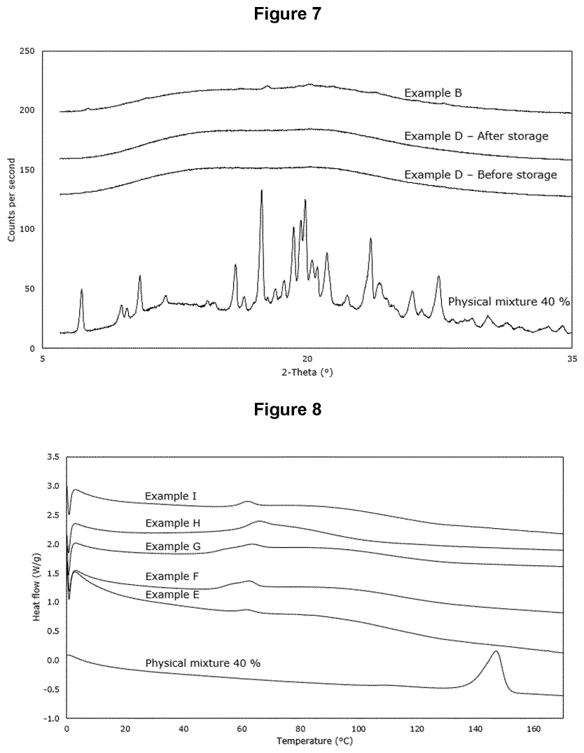

### FIG. 7

FIG. 7 shows pXRD measurements of Examples B after preparation and D before and after storage compared to a physical mixture containing the polymer and 40% (w/w) of ketoconazole.

Graphs show that formulation prototypes of Example D were amorphous after preparation and after storage. Formulation prototypes of Example B shows traces of crystallinity.

### FIG. 8

FIG. 8 shows DSC thermograms of Examples E-I in comparison to a physical mixture of polymer and ketoconazole. Displayed is the first heating cycle. No melting events were observed for the prepared formulation types indicating they were amorphous after preparation.

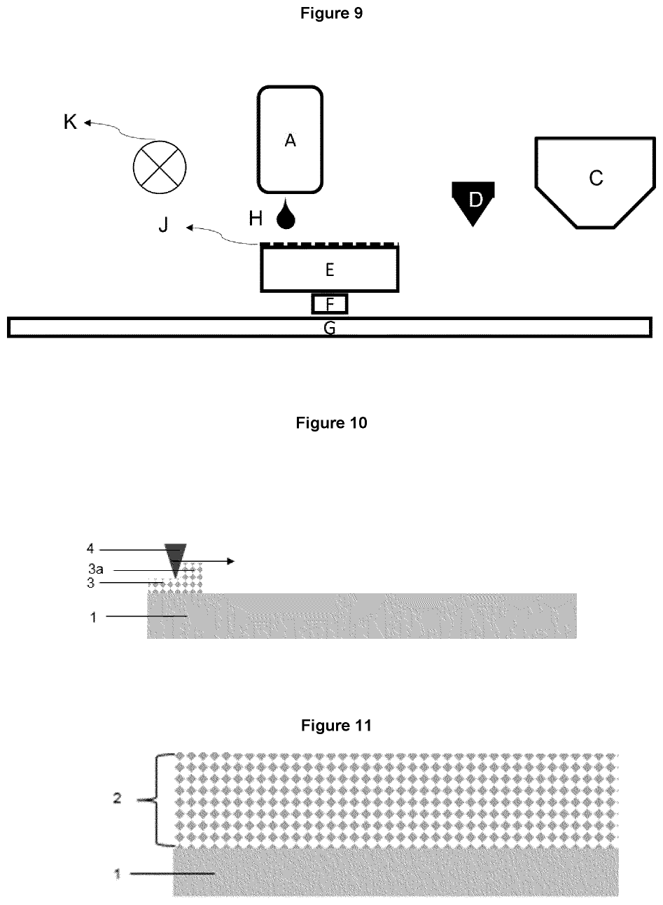

### FIG. 9

FIG. 9 shows a scheme of the printing apparatus. The build plate (E) is connected to a control system. By means of the axes the build plate can be moved to different locations aligning it with additional parts of the printing apparatus. For the manufacturing of a single layer the build plate is moved to the powder supply (C) where powder is applied to the surface of the build plate or the surface of the powder bed. A blade (D) is used to spread a thin layer of powder (J) while the build plate is moved under the blade in a linear motion. A jet printing assembly (A) can be used to apply droplets of fluids (H) onto the surface of the powder layer in a spatially controlled manner. A halogen lamp (K) can be used to irradiate the powder bed.

### FIG. 10

FIG. 10 illustrates the spreading step (a) of the process. Onto a mounting plate (1) a powder provided by a powder reservoir (3a) is spread by moving a doctor blade (4) in the direction indicated by an arrow to achieve a powder layer. A part of the powder layer that is already spread is indicated by (3). By repeating the spreading of powder on the already existing powder layer(s) as often as necessary a powder bed is created.

### FIG. 11

FIG. 11 shows the powder bed (2) that is created step by step (a) on the mounting plate (1).

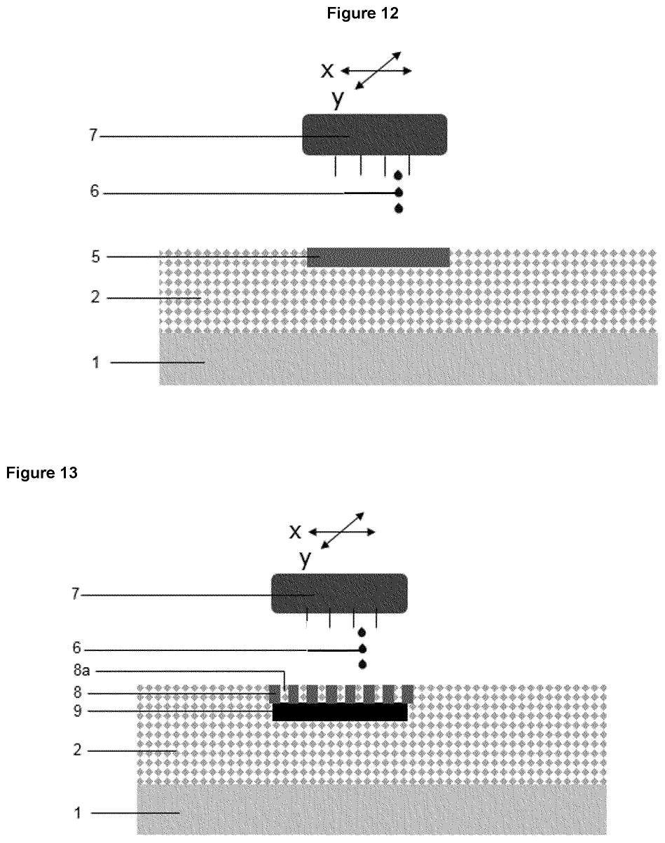

### FIG. 12

FIG. 12 shows jet printing in accordance to step (c) of the process. A jet head (7) is moved along x and/or y axis thereby jet printing a fluid (6) (in fine droplets) onto the powder bed (2). Such jet printing results in powder soaked with fluid (5) created by voxels that are adjacent to one another.

### FIG. 13

FIG. 13 shows the jet printing step as in FIG. 9 whereby the intermediate product shown in FIG. 9, onto which a layer of powder was spread, is used. In contrast to FIG. 9 the fluid is not jet printed on a continuous area but on defined areas of the powder that are delimited from each other so that a layer of powder voxels soaked with fluid (8) and powder voxels without fluid (8a) are created.

1. A process for the manufacture of a solid pharmaceutical administration form comprising an active ingredient comprising the steps:
(a) preparing a powder comprising particles of an amorphous solid dispersion of an active ingredient in a polymeric matrix;
(b) spreading the powder prepared by step (a) across the manufacturing area to create a powder bed;
(c) jet printing a medium onto the powder whereby such medium is suitable to provide binding of the powder;
(d) spreading a layer of powder prepared by step (a) onto the surface of powder obtained after performing step (c) and subsequently performing step (c);
(e) repeating step (d) as often as needed to build up the solid pharmaceutical administration form; and
(f) separating the solid pharmaceutical administration form from the powder bed.

2. The process for the manufacture of a solid pharmaceutical administration form according to claim 1, wherein the amorphous solid dispersion of an active ingredient in a polymeric matrix, which is present as particles in the powder prepared by step (a), is prepared using hot melt extrusion, co-precipitation or spray drying.

3. The process for the manufacture of a solid pharmaceutical administration form according to claim 1, wherein the medium used for jet printing in step (c) is a liquid.

4. The process for the manufacture of a solid pharmaceutical administration form according to claim 1, wherein the powder comprises a binding material.

5. The process for the manufacture of a solid pharmaceutical administration form according to claim 4, wherein the medium is a liquid which partly dissolves at least one of the polymeric matrix and the binding material.

6. The process for the manufacture of a solid pharmaceutical administration form according to claim 5, wherein the medium consists of one or more volatile solvent(s).

7. The process for the manufacture of a solid pharmaceutical administration form according to claim 5, wherein the medium comprises one or more volatile solvent(s) in an admixture with one or more non-volatile solvent(s).

8. The process for the manufacture of a solid pharmaceutical administration form according to claim 1, wherein the medium is a fluid and comprises a binding material.

9. The process for the manufacture of a solid pharmaceutical administration form according to claim 8, wherein the binding material comprises lactose, sorbitol, mannitol, xylitol, maltitol, glucose, fructose, sucrose, sucrose fatty acid esters (e.g. sucrose stearate, sucrose palmitate), sorbitan esters (e.g. Span®), glycerol fatty acid esters (e.g. glycerol monostearate), fatty acids, fatty alcohols, esters of fatty acids with fatty alcohols and polymers (e.g. polyvinylpyrrolidone, polyvinyl alcohol, polyvinyl acetate, a vinylpyrrolidone-vinyl acetate copolymer, polyethylene glycol), a starch (e.g. maize starch, pre-gelatinized starch), a cellulose derivative (e.g. hydroxypropyl methylcellulose, hydroxypropyl cellulose, ethyl cellulose, hydroxypropyl methylcellulose acetate succinate, microcrystalline cellulose), a copolymer of acrylic or methacrylic acid and an acrylic or methacrylic ester (e.g. a copolymer of methacrylic acid and a methacrylate or a acrylate, such as Poly(methacrylic acid-co-methyl methacrylate) (1:1) (e.g. Eudragit® L 100), Poly(methacrylic acid-co-methyl methacrylate) (1:2) (e.g. Eudragit® S 100) or Poly(methacrylic acid-co-ethyl acrylate) (1:1) (e.g. Eudragit® L 100-55)), a copolymer of ethyl acrylate, methyl methacrylate and a low content of methacrylic acid ester with quaternary ammonium groups (e.g. Poly(ethyl acrylate-co-methyl methacrylate-co-trimethylammonioethyl methacrylate chloride) 1:2:0.2 (e.g. Eudragit® RL), Poly(ethyl acrylate-co-methyl methacrylate-co-trimethylammonioethyl methacrylate chloride) 1:2:0.1 (e.g. Eudragit® RS)), or a copolymer of dimethylaminoethyl methacrylate, butyl methacrylate, and methyl methacrylate, (e.g. Poly(butyl methacrylate-co-(2-dimethylaminoethyl) methacrylate-co-methyl methacrylate) (2:1:1) (e.g. Eudragit® E PO)).

10. The process for the manufacture of a solid pharmaceutical administration form according to claim 1, wherein the polymeric matrix comprises a vinylpyrrolidone-vinyl acetate copolymer (PVP-VA), polyvinylpyrrolidone (PVP), polyvinyl alcohol (PVA), hydroxypropyl methylcellulose acetate succinate (HPMCAS), Poly(methacrylic acid-co-ethyl acrylate) (e.g. Eudragit® L100-55), poly(methacrylic acid-co-methyl methacrylate) (e.g. Eudragit® L and Eudragit® S), polyvinyl caprolactam-polyvinyl acetate-polyethylene glycol graft copolymer (PVCap-PVAc-PEG) (e.g. Soluplus®), hydroxypropyl methylcellulose phthalate (HPMCP), cellulose acetate phthalate (CAP), polyvinyl acetate phthalate (PVAP), cellulose acetate trimellitate (CAT) or hydroxypropyl methylcellulose acetate trimellitate (HPMCAT).

11. The process for the manufacture of a solid pharmaceutical administration form according to claim 1, wherein a drying step is performed after performing at least one of step (c) and step (d).

12. The process for manufacture of a solid pharmaceutical administration form according to claim 1, wherein the pharmaceutical administration form is for oral use.

13. The process for manufacture of a solid pharmaceutical administration form according to claim 12, wherein the pharmaceutical administration form provides immediate release of the active pharmaceutical ingredient.

14. The process for manufacture of a solid pharmaceutical administration form according to claim 1, wherein a drying step is performed after step (f).

15. The process for the manufacture of a solid pharmaceutical administration form according to claim 6, wherein the one or more volatile solvent(s) is selected from the group consisting of methanol, ethanol, propanol, 2-propanol and acetone.

16. The process for the manufacture of a solid pharmaceutical administration form according to claim 7, wherein the one or more volatile solvent(s) is selected from the group consisting of methanol, ethanol, propanol, 2-propanol and acetone and the one or more non-volatile solvent(s) is selected from the group consisting of water, N-methyl-2-pyrrolidone, N-ethyl-2-pyrrolidone, dimethylformamide, dimethylacetamide and dimethylsulfoxide

17. The process for the manufacture of a solid pharmaceutical administration form according to claim 8, wherein the binding material is selected from the group consisting of polyvinyl alcohol and hydroxypropyl methylcellulose acetate succinate.

18. The process for the manufacture of a solid pharmaceutical administration form according to claim 8, wherein the binding material comprises hydroxypropyl methylcellulose acetate succinate.

19. The process for the manufacture of a solid pharmaceutical administration form according to claim 4, wherein the binding material comprises lactose, sorbitol, mannitol, xylitol, maltitol, glucose, fructose, sucrose, sucrose fatty acid esters (e.g. sucrose stearate, sucrose palmitate), sorbitan esters (e.g. Span®), glycerol fatty acid esters (e.g. glycerol monostearate), fatty acids, fatty alcohols, esters of fatty acids with fatty alcohols and polymers (e.g. polyvinylpyrrolidone, polyvinyl alcohol, polyvinyl acetate, a vinylpyrrolidone-vinyl acetate copolymer, polyethylene glycol), a starch (e.g. maize starch, pre-gelatinized starch), a cellulose derivative (e.g. hydroxypropyl methylcellulose, hydroxypropyl cellulose, ethyl cellulose, hydroxypropyl methylcellulose acetate succinate, microcrystalline cellulose), a copolymer of acrylic or methacrylic acid and an acrylic or methacrylic ester (e.g. a copolymer of methacrylic acid and a methacrylate or a acrylate, such as Poly(methacrylic acid-co-methyl methacrylate) (1:1) (e.g. Eudragit® L 100), Poly(methacrylic acid-co-methyl methacrylate) (1:2) (e.g. Eudragit® S 100) or Poly(methacrylic acid-co-ethyl acrylate) (1:1) (e.g. Eudragit® L 100-55)), a copolymer of ethyl acrylate, methyl methacrylate and a low content of methacrylic acid ester with quaternary ammonium groups (e.g. Poly(ethyl acrylate-co-methyl methacrylate-co-trimethylammonioethyl methacrylate chloride) 1:2:0.2 (e.g. Eudragit® RL), Poly(ethyl acrylate-co-methyl methacrylate-co-trimethylammonioethyl methacrylate chloride) 1:2:0.1 (e.g. Eudragit® RS)), or a copolymer of dimethylaminoethyl methacrylate, butyl methacrylate, and methyl methacrylate, (e.g. Poly(butyl methacrylate-co-(2-dimethylaminoethyl) methacrylate-co-methyl methacrylate) (2:1:1) (e.g. Eudragit® E PO)).

20. The process for the manufacture of a solid pharmaceutical administration form according to claim 4, wherein the binding material is selected from the group consisting of polyvinyl alcohol and hydroxypropyl methylcellulose acetate succinate.
